(19) United States
(12) Patent Application Publication
(10) Pub. No.: US 2010/0203026 A1
(43) Pub. Date: Aug. 12, 2010

(54) PROBIOTICS FOR INDUCING SATIETY AND/OR SATIATION

(75) Inventors: Marta Zdzislawa Korczynska, Wageningen (NL); Wouter Noordman, Ede (NL); Arthur Constantijn Ouwehand, Inga (FI)

Correspondence Address: BROWDY AND NEIMARK, P.L.L.C.;624 NINTH STREET, NW, SUITE 300, WASHINGTON, DC, 20001-5303, US

(73) Assignee: CAMPINA NEDERLAND HOLDING B.V., ZALTBOMMEL (NL)

(21) Appl. No.: 12/670,537

(22) PCT Filed: Jul. 25, 2007
(86) PCT No.: PCT/NL07/50369

§ 371 Date: Apr. 23, 2010

(51) Int. Cl. A61K 35/74; C12N 1/20; C12Q 1/02; A61P 3/04

(52) U.S. Cl. 424/93.45; 435/252.1; 435/253.6; 435/252.9; 424/93.4; 435/29

(57) ABSTRACT

The invention relates to the field of nutrition and medicine. Provided are compositions, bacterial strains and methods for inducing or enhancing satiety and satiation and for treating or preventing obesity, overweight and overweight related diseases.

### FIELD OF THE INVENTION

The instant invention relates to the field of nutrition, especially to new probiotic strains, compositions comprising at least one of these, or comprising cell-free medium obtainable from culture medium (especially fermentation medium) of at least one of these strains, and methods for making and/or using such compositions and/or strains and/or cell-free culture medium. The compositions are suitable for inducing satiety and/or satiation and, thus, for causing weight loss and/or preventing and/or treating weight gain and overweight related diseases such as obesity and related heart diseases.

### BACKGROUND OF THE INVENTION

It is known that preparations of natural fats such as Fabuless™ (DSM) and PinnoThin™ (Lipid Nutrition), have a satiating effect when ingested. PinnoThin contains the polyunsaturated fatty acid pinolenic acid, derived from the seeds of the Korean pine nut tree (Pinus koraiensis) and which enhances expression of the appetite-suppressing hormones (glucagon-like peptide-1) GLP-1 and cholecystokinin (CCK). These hormones are related to phenomena of satiety, satiation) (reference: C. de Graaf et al, Am. J. Clin Nutr (2004) 79: 946-61. Fabuless™ contains palm and oat oil and on digestion the body identifies a relatively high level of undigested fat at a relatively late stage of the digestive process, whereby the hunger signals are suppressed or delayed.

Thus, certain macronutrients, such as fats, enhance the secretion of satiety hormones, such as CCK and GLP-1. CCK is released in the duodenum in the presence of fats and proteins. GLP-1 is released by intestinal L-cells in response to carbohydrates and fats, although its secretion may also be stimulated by the central nervous system. The hormones GLP-1 and protein peptide Y (PYY), both of which are secreted by enteroendocrine L-cells of the small and large intestine, may play a role in the “ileal break” mechanism and are involved in the control of gastrointestinal secretion and motility. The hormone ghrelin is produced in the fundus of the stomach and its synthesis can be suppressed by carbohydrates, thereby reducing the feeling of hunger.

WO2007/043933 describes the use of three well known probiotic strains (Lactobacillus casei F19-LMG P-17806; Lactobacillus acidophilus NCFB 1748 and Bifidobacterium lactis Bb12 DSM 10140) for influencing fat metabolism and obesity. These known probiotic strains were not identified based on a functional assay designed to identify strains which modulate satiety and/or satiation, but appear to be chosen due to their effect on weight reduction as a secondary parameter in a human trial concerning cholesterol lowering. Also, the strains were added to acidified milk, i.e. to the final test product, without allowing for fermentation to take place. Moreover, the instant invention relates to strains which modulate the induction of satiety hormone levels directly and which are, therefore, highly effective at enhancing satiety and/or satiation after consumption.

There remains a need in the art for identifying LABs and LAB-derived metabolites which are capable of causing a pronounced reduction in food or feed intake and which are effective in significantly reducing food or feed intake. Such LABs, LAB-derived metabolites and compositions comprising these can be used to treat and/or prevent weight gain, obesity, overweight related diseases (heart diseases, strokes, diabetes, etc.) and cause weight loss.

### DEFINITIONS

“Lactic acid bacteria” (LAB) and “lactic acid producing bacteria”, is used herein interchangeably and refers to probiotic bacteria, which produce lactic acid or another organic acid (such as propionic acid) as an end product of fermentation, such as, but not limited to, bacteria of the genus Lactobacillus, Streptococcus, Lactococcus, Oenococcus, Leuconostoc, Pediococcus, Carnobacterium, Propionibacterium, Enterococcus and Bifidobacterium.

“Probiotics” or “probiotic strain(s)” refers to strains of bacteria, which have a beneficial effect on the host when ingested by a subject and which are generally regarded as safe (GRAS) to humans.

“Supernatant” is a cell-free medium obtainable after removing bacterial cells from the culture medium (such as preferably the fermentation broth) by techniques such as filtration or centrifugation. The supernatant can be fractionated or concentrated before use. “Supernatant” will be equivalently called thereafter “cell-free medium”.

Fermentation medium” and “fermentation broth” are used herein interchangeably to refer to either the medium to which the bacterial strains are added (i.e. medium without the strain/s) or the medium (broth) during and/or after fermentation by the added bacteria (i.e. comprising fermentation products), depending on the context. It will be clear from the context which meaning the term has.

A “subject” refers herein to a human or non-human animal, in particular a vertebrate.

“Modulation of food intake” or “reduction in food intake” refers to a significant decrease in food intake as a result of satiety and/or satiation.

“Satiety” or “enhanced satiety” refers to the prolonged feeling of fullness after a meal, and a prolonged period of time until the next meal.

“Satiation” or “enhanced satiation” refers to earlier meal termination due to an earlier feeling of fullness.

“Satiety related hormones” or “satiety hormones” refer to hormones which, when induced or repressed, have an effect on satiety and/or satiation, such as, but not limited to, CCK, and/or GLP-1, bombesin, somatostatin, insulin, leptin, glucose-dependent insulinotropic polypeptide (GIP), ghrelin, Peptide YY (PYY), enterostatin.

“Hormone induction” refers to the induction (stimulation) of gastrointestinal hormone synthesis, such as GLP-1 and/or CCK levels, which can be measured in the blood plasma and/or in in vitro cell-line assays.

“Hormone repression” refers to the reduction of gastrointestinal hormone synthesis, such as ghrelin levels, which can be measured in the blood plasma and/or in in vitro cell-line assays.

“In vitro cell lines” and “in vitro cell line assay” refers to the use of cell-line cultures, especially mammalian cell lines capable of producing gastrointestinal hormones, such as CCK and/or GLP-1, for identifying probiotic strains and/or for determining the functionality of probiotic strains in their ability to cause hormone induction and/or repression.

“Metabolism” are the chemical reactions occurring in living cells. “Metabolites” are molecules made as a result of metabolism, and include herein metabolites made during catabolic reactions (reactions yielding energy) and anabolic reactions (reactions using energy). Metabolites include, thus, also compounds in the culture medium of the cells (e.g. in the cell-free medium), such as degradation products of one or more components of the culture medium/growth medium/growth environment or specific components synthesized by the bacterial cells.

“Percentage” or “average” generally refers to percentages of averages by weight, unless otherwise specified or unless it is clear that another basis is meant.

The term “comprising” is to be interpreted as specifying the presence of the stated parts, steps or components, but does not exclude the presence of one or more additional parts, steps or components.

The term “a” or “an” does not limit to one, but is interpreted as at least one.

### DETAILED DESCRIPTION

The inventors found that in vitro cell line assays can be used efficiently to identify and/or isolate (probiotic) bacterial strains, which significantly induce satiety and/or satiation in human and/or mammalian subjects. By screening strains which are capable of stimulating gastrointestinal satiety hormone production, such as CCK and/or GLP-1, in an in vitro cell line assay, strains could be identified which are capable of inducing gastrointestinal satiety hormone production, such as CCK and/or GLP-1 production. Without limiting the invention, it is believed that the strains induce (or significantly enhance or stimulate) a feeling of satiety and/or satiation by stimulating gastrointestinal satiety hormone production in vivo (following ingestions of a suitable amount of a composition comprising one or more such strains and/or of cell-free culture medium obtained/obtainable from culturing one or more such strains, especially obtained/obtainable from culturing the strains under fermentation conditions).

### Screening Methods According to the Invention

In one embodiment a method for identifying and/or isolating bacteria which are capable of inducing or enhancing satiety and/or satiation is provided, comprising the steps of:
(a) culturing one or more bacterial strains on a medium,
(b) collecting the bacterial cells and/or bacterial cells with culture medium and/or cell-free culture medium of (a),
(c) adding the bacterial cells and/or bacterial cells and culture medium and/or cell-free medium to a mammalian cell line culture,
(d) analyzing the amount of satiety hormone (e.g. GLP-1 and/or CCK) produced by the cell line after addition of (c), and
(e) identifying the bacterial strain(s) which caused (directly or indirectly, via the culture medium or metabolites present in the medium) a (preferably significantly) larger amount of satiety hormone (e.g. GLP-1 and/or CCK) being produced following treatment with bacterial cells and/or bacterial cells with culture medium and/or cell-free culture medium compared to controls (untreated mammalian cell line, mammalian cell line treated with control samples, such as buffer only or another medium lacking the bacteria), and
(f) optionally, using the identified bacterial strains and/or bacterial cells with culture medium and/or cell-free culture medium obtained/obtainable from culturing the identified strains, for the preparation of a composition for reducing food intake, inducing and/or enhancing satiety and/or satiation, treatment or prevention of obesity and/or for causing weight loss.

In step (a) any bacterial strain may be used, but preferably strains are probiotic strains, more preferably strains belonging to the LAB are used. The culture medium is preferably a medium on which growth and/or fermentation can take place (fermentation medium), such as a dairy product or milk based medium (e.g. skimmed milk, etc.), or ‘milks’ of vegetable origin (e.g. soybean milk), although also other media are suitable. Thus, substrates should be available which allow products of fermentation, such as lactic acid and/or other organic acids, to be made. The culturing conditions (fermentation time) should allow the fermentation process to take place, i.e. the incubation time and temperatures, etc. are selected accordingly, as known in the art. Typical fermentation times are several hours to several days. During fermentation the pH drops, due to the production of organic acids. Optionally, the pH may be adapted at the end of fermentation, e.g. by further addition of one or more organic acids (such as lactic acid) or a pH buffering substance. Optionally, the fermentation medium may be supplemented with prebiotics, protein hydrolysates (e.g. casaminoacids), peptones, minerals, vitamins, yeast extract, etc.

In step (b) the bacterial cells and/or culture medium with bacterial cells are/is collected. Collection can take place e.g. by centrifugation and/or filtration and/or concentration, and the cells (and/or medium with cells) can be resuspended in a suitable buffer for treatment of the mammalian cell-line culture (in step (c)). Preferably, the bacterial cells are diluted to an appropriate concentration, such as a concentration which does not have negative effects on the mammalian cell line as such. Dilution series can be used to test which concentration of cells (CFU) is most appropriate. The bacterial cells may optionally be washed prior to use (e.g. to remove all of the medium), but in one embodiment, the collected bacterial cells are preferably not washed after collection and prior to re-suspension or contact with the mammalian cell line. Thus, in one embodiment, the cells are collected (e.g. by centrifugation and/or filtration and/or concentration) and are used directly to make a suspension or dilution and are then brought into contact with the mammalian cell line.

Alternatively, the cell-free medium, obtained by removal of (all or substantially all of) the bacterial cells, e.g. by centrifugation or filtration, or one or more fractions obtained thereof, are collected and used in (c). In order to identify which component of the cell-free medium (e.g. which metabolite/s) is capable of stimulating satiety hormone production (e.g. GLP-1 and/or CCK production) partially or substantially purified fractions of the cell-free medium may be used. Fractions which retain satiety hormone inducing activity can then eventually be used to identify one or more specific components having satiety hormone inducing activity.

Another alternative is to use a combination of bacterial cells and culture medium (e.g. fermentation broth) as such, optionally concentrated or diluted.

In step (c), an in vitro assay is carried out to test whether the bacterial cells and/or bacterial cells with medium and/or cell-free medium (or fractions thereof) have a satiety hormone inducing effect on mammalian cells grown in culture. A number of mammalian cell lines are available in the art which can be used. In a preferred embodiment the line used is STC-1 (native intestinal endocrine cells from endocrine tumor of the small bowel of transgenic mice), as for example described by McLaughlin et al. (1998) J Physiol 513:11-18) or by the following publications:

Gevrey et al. Diabetologia (2004) 47:926-936; Cordier-Bussat et al. Endocrinology 138:1137-1144 (1997); Hira et al. J. Biol. Chem. (2004) 279 (25) 26082-26089; Kazmi et al. J. Physiol (2003), 553:759-773; Trinh et al Diabetes (2003) 52: 425-433; Benson et al. J. Physiol. (2002) 538:121-131; Wang et al. Am J Physiol Gastrointest Liver Physiol (2002) 282:16-22; CordierBussat et al. Diabetes (1998) 47:1038-1045.

Other suitable cell lines include for example the line GLUTag (intestinal endocrine tumour cell line from the large bowel of transgenic mice) (see Brubaker et al. Endocrinology (1998)139: 4108-4114, 1998; Reimann et al. Diabetologia (2004) 47: 1592-1601; Reimann et al. J Physiol (2005) 563:161-175; Reimann et al. J Physiol (2005) 569):761-772; Anini et al. Diabetes. (2003) 52:252-259; and Reimann et al. Diabetes (2002) 51:2757-63) NCI-H716 (human intestinal cell line) (see Anini et al. Endocrinology (2003) 144:3244-3250; Cao et al. Endocrinology (2003) 144:2025-2033; Anini et al. Diabetes (2003) 52:252-259; Reimer et al. Endocrinology (2001) 142:4522-4528), FRIC (fetal rat intestinal cell line) (see Brubaker et al. Endocrinology. (1991) 129:3351-8; and Anini et al. Endocrinology (2002) 143:2420-2426), isolated canine L-cells (see Damholt et al. Endocrinology (1998) 139:2085-2091), Caco-2 cells (see Hyae Gyeong Cheon Biochem Pharmacol. (2005) 70:22-29), and others. The skilled person can identify suitable cells lines, which are capable of producing one or more satiety hormones, such as GLP-1 and/or CCK (base level and/or after induction).

Step (d) can be carried out in various ways. For example the hormones like the GLP-1 and/or CCK proteins (or ‘peptides’, which is used interchangeably herein to refer to amino acid chains) can be identified and optionally quantified using e.g. ELISA (i.e. using antibody based screens for detecting the proteins) or other methods. Alternatively, gene expression (mRNA transcription) can be analysed using methods known in the art, such as quantitative RT-PCR, or other molecular biology methods. The human GLP-1 peptide has the amino acid sequence of SEQ ID NO: 1 (hdeferhaeg tftsdvssyl egqaakefia wlvkgrg), see GenBank Accession No. P01275. The mouse homolog is identical to the human peptide, see GenBank P55095. The rat CCK protein precurser has Accession No. P01355 (SEQ ID NO: 2, mkcgvcicvv mavlaagala qpvvpveavd pmeqraeeap rrqlravlrp dseprarlga llaryiqqvr kapsgrmsvl knlqgldpsh risdrdymgw mdfgrrsaed yeyps) and mouse P09240 (SEQ ID NO: 3, mkcgvcicvv mavlaagala qpvvpaeatd pveqraeeap rrqlravlrp dreprarlga llaryiqqvr kapsgrmsvl knlqsldpsh risdrdymgw mdfgrrsaed yeyps).

Thus, both the protein and cDNA sequences are available in the art, as are antibodies capable of detecting the proteins (or homologs thereof, such as proteins comprising at least 80, 90, 95, 98% or more amino acid sequence identity to SEQ ID NO: 1, 2 or 3, as determined using e.g. pairwise alignments such as the program ‘needle’ or ‘water’ of EmbossWin, with gap opening penalty 10.0 and gap extension penalty 0.5, using the Blosum62 substitution matrix) and methods for detecting relative or absolute levels of satiety hormones such as the GLP-1 and/or CCK proteins. See also the above references on suitable mammalian cell lines, which also measure GLP-1 and/or CCK release following stimulation with e.g. peptone, fatty acids or other agents.

The absolute or relative amount of satiety hormones, such as GLP-1 and/or CCK transcript (mRNA or corresponding cDNA) and/or protein produced following treatment is compared to suitable controls, in order to determine whether or not transcription and protein production was induced by the treatment. Suitable controls include, for example, i) untreated mammalian cells (for determining e.g. the base levels of GLP-1 and/or CCK), ii) mammalian cells treated with buffer lacking the bacterial cells and/or lacking the bacterial cells with medium and/or lacking the cell-free medium or iii) mammalian cells treated with control medium (e.g. skimmed milk acidified e.g. by lactic acid).

Step (e) optionally uses statistical analysis to identify those bacterial strains who (directly or indirectly via the medium, e.g. via one or more metabolites present in the medium) resulted in a significantly increased production of one or more satiety hormones, e.g. GLP-1 and/or CCK, compared to the control. A ‘significant increase’ refers herein to at least 1,5×, preferably at least 2×, 3×, 4×, 5×, 6× or more of the amount produced in the control.

Various steps can be repeated, if desired. For example, mixtures of two or more bacterial cell cultures and/or bacterial cells with medium and/or cell-free media (or various fractions thereof) may be used, in order to test the satiety hormone inducing activity of the mixtures. Also, different mammalian cell lines may be tested for satiety hormones like GLP-1 and/or CCK induction following treatment.

Strains obtained by the above method are also an embodiment of the invention, as are compositions comprising one or more of such strains (with or without culture medium, e.g. fermentation medium) and/or comprising one or more of the cell-free medium of such strains (or parts thereof), such as the fermentation medium (or parts thereof).

Step (f) is optional and makes use of the identified bacterial strains and/or bacterial cells with culture medium and/or cell-free culture medium obtained/obtainable from culturing the identified strains for making compositions. Initially, compositions comprising one or more of these may be made and fed to animals, such as rats, in order to validate the satiety and/or satiation inducing effect in vivo, preferably followed by human trials. Preferably, food/feed intake is reduced following ingestion. The effect may be seen as an earlier meal termination compared to control food/feeds and/or longer intervals between meals/feeding. The effect is also seen as a prolonged period until there is a desire for a next meal. Food intake/feeding for extended periods using food/feed compositions comprising the bacteria and/or comprising the bacteria and culture medium and/or comprising cell-free medium (preferably medium obtained as a result of fermentation) may also result in a lower body weight compared to the control food/feed compositions.

In human subjects also subjective ratings before and at certain time intervals after ingestion of the composition(s) can be assessed (e.g. subjective hunger, fullness, satiety, desire to eat, etc.) using, for example, the Visual Analogue Scale (VAS), as known in the art (see e.g. Luscombe-Marsh et al., Am. J. Clin. Nutr. (2005) 81:762-772 or Moran et al., J. Clin. Endocrinol. Metabol. (2005)90:5205-5211). Subjects are asked to make a mark on each scale between the extremes (e.g. hungry to not hungry) to indicate their feelings at that time. The change in ratings from baseline is then quantified.

### Strains and Culture Media According to the Invention

In one embodiment the invention provides (probiotic) bacterial strains, which are capable of stimulating satiety and/or satiation following administration by a human and/or animal subject (e.g. by ingestion).

The bacterial strains used are preferably lactic acid or other organic acid e.g. propionic acid producing probiotic bacteria, preferably of the genus Lactobacillus. Also preferred are bacteria of the genus Bifidobacterium or of the genus Propionibacterium. The bacteria should be food-grade, i.e. they should be considered as not harmful, when ingested by a human or animal subject. It is understood that non-food grade bacteria, for example pathogenic bacteria, which have been modified so that they are no longer harmful when ingested by a subject, are included within the scope of the invention.

Preferably the strains belong to the genus Lactobacillus. The Lactobacillus strains may be of the following species: L. rhamnosus, L. casei, L. paracasei, L. helveticus, L. delbrueckii, L. reuteri, L. brevis, L. crispatus, L. sakei, L. jensenii, L. sanfransiscensis, L. fructivorans, L. kefiri, L. curvatus, L. paraplantarum, L. kefirgranum, L. parakefir, L. fermentum, L. plantarum, L. acidophilus, L. johnsonii, L. gasseri, L. xylosus, L. salivarius etc. Preferred species are L. delbrueckii ssp bulgaricus, L. casei, L. acidophilus, L. paracasei, L. helveticus, L. fructivorans, L. salivarius, L. curvatus, and L. sakei.

The species identity of micro-organisms can be determined biochemically or by DNA sequencing (e.g. conserved regions) or by known methods such as pulse field gel electrophoresis. In general, strains of bacteria belong to the same species if they show at least 97% nucleic acid sequence identity in the 16 S rRNA region (e.g. when optimally aligned by for example the programs GAP or BESTFIT using default parameters).

The bacterial strain are capable of stimulating satiety hormone, e.g. glucagon-like peptide-1 (GLP-1) and/or cholecystokinin (CCK), production and/or release in an in vitro cell-line assay and/or in subjects in vivo. In in vitro cell-line assays, the capability to induce satiety hormones (e.g. GLP-1 and/or CCK) production can be tested as described above. In in vivo experiments in humans or mammals, the production/induction of satiety hormones such as GLP-1 and/or CCK can be compared by measuring the blood plasma levels of these proteins before and at one or more time points after administration or ingestion of the strain(s) or compositions comprising one or more strains according to the invention and/or cell-free culture medium (or fractions thereof) according to the invention. As mentioned above, the strains can be characterized by their ability to cause (directly, or indirectly via the culture medium) a significant increase in hormone production, in particular GLP-1 and/or CCK production, relative to the control treatments and/or the base level production.

Of course, the strains can also be characterized by their satiety and/or satiating effect on subjects. Thus one or more strains can be grown up and harvested using standard methods. Alternatively, the cell-free culture medium can be used, which may comprise one or more satiety modulating metabolites. Various dilutions of the bacterial cells and/or the cell-free medium (or fractions thereof) can be made in a suitable medium, such as a buffer, water, a food base (e.g. skimmed milk, etc.) to make test compositions. These can then be administered (preferably orally) to the test animals and/or human subjects, using experimental setups which preferably allow the appropriate statistical analysis to be carried out (e.g. double blind, placebo controlled trials). The effect of the test composition on satiety can be determined by scoring for example the period of fullness until the next meal. A satiety effect is seen if the period of the feeling of fullness is significantly longer than in the controls, such as for example at least 1%, 2%, 3%, 5%, 10%, 20% longer, or more. In addition or alternatively the satiety effect can be determined by measuring the amount of food consumed (until a pleasant feeling of fullness is reached) during a meal that is taken after a certain period (e.g. 4 hours) following consumption of product containing the active ingredient (e.g. the test composition). A satiety effect is seen if the amount of calories consumed is at least 1%, 2%, 3%, 4%, 5% or 10% or 15% (or a higher number) less than consumed after eating a control product without the active ingredient.

The effect on satiation can be determined by scoring the time point of meal termination. A satiation effect is seen if the amount of consumed calories at meal termination is significantly less than in the controls, such as for example at least 1%, 2%, 3%, 4%, 5%, 10% 20%, or more. Over a longer time period (such as 1, 2, 3, 4, 5 weeks or more), one can also score the body weight reduction or the body weight change compared to a control diet. Body weight of a subject being administered regular amounts of the test compositions (e.g. once daily, twice daily, or more) is preferably significantly controlled (reduced or less increased) compared to the control subjects.

In a preferred embodiment the following strains (and replicates or derivatives thereof) are provided, as well as compositions comprising one or more of these and/or as well as compositions comprising culture medium obtained/obtainable from growing one or more of these (e.g. under fermentation conditions): Lactobacillus acidophilus MUH 41, Lactobacillus delbrueckii ssp bulgaricus MUH 192, Lactobacillus casei ssp paracasei MUH 142, Lactobacillus delbrueckii ssp bulgaricus MUH 190 have been deposited by Campina Nederland BV, The Netherlands at the Centraalbureau voor Schimmelcultures (CBS, Uppsalalaan 8, 3584 CT Utrecht, The Netherlands) on 5 Jul. 2007 and have been assigned Accession numbers CBS 121540 (L. acidophilus MUH 41), CBS 121543 (L. delbrueckii ssp bulgaricus MUH 192), CBS 121541 (L. casei ssp paracasei MUH 142) and CBS 121542 (L. delbrueckii ssp bulgaricus MUH 190); Lactobacillus acidophilus NCFM deposited by Rhodia Chimie, France, at the American Type Culture Collection as PTA-4797 on Nov. 15, 2002; Lactobacillus salivarius 33 deposited by Rhodia Chimie, France, at the American Type Culture Collection as PTA-4800 on Nov. 15, 2002; Lactobacillus salivarius 1502 deposited by Danisco Niebüll GmbH at the Deutsche Sammlung von Mikroorganismen and Zellkulturen GmbH as DSM 15794 on Jul. 23, 2003; Lactobacillus curvatus 853 deposited Danisco Niebüll GmbH at the Deutsche Sammlung von Mikroorganismen and Zellkulturen GmbH as DSM 15793 on Jul. 23, 2003, and Lactobacillus sakei 570 deposited by Danisco A/S at the Deutsche Sammlung von Mikroorganismen and Zellkulturen GmbH as DSM 15889 on Sep. 2, 2003. These strains and/or derivatives and/or replicates of these (and/or medium obtainable from culturing these) are suitable for making compositions which reduce food/feed intake.

It is understood that replicates and/or derivatives of the deposited strains or any other strain according to the invention are encompassed by the invention. The term “replicate” refers to the biological material that represents a substantially unmodified copy of the material, such as material produced by growth of micro-organisms, e.g. growth of bacteria in culture media. The term “derivative” refers to material created from the biological material and which is substantially modified to have new properties, for example caused by heritable changes in the genetic material. These changes can either occur spontaneously or be the result of applied chemical and/or physical agents (e.g. mutagenesis agents) and/or by recombinant DNA techniques as known in the art. When referring to a strain “derived” from another strain, it is understood that both “replicates” of that strain, as well as “derivatives” of the strain are encompassed, as long as the derived strain still retains the reducing effect on food/feed intake.

In one embodiment the strains are not strains selected from the group consisting of Lactobacillus casei F19-LMG P-17806, Lactobacillus acidophilus NCFB 1748 and Bifidobacterium lactis Bb-12 DSM 10140.

### Compositions According to the Invention

The invention also provides compositions comprising a satiating and/or satiety inducing amount of one or more bacterial strains and/or bacterial cells with medium, preferably after fermentation, and/or cell-free medium derived thereof.

When referring to “supernatants” or “culture supernatants” or “cell-free medium” herein, it is clear that the medium in which the bacteria, here preferably lactic acid bacteria, are multiplied (grown/cultured) is referred to, preferably the fermentation medium, but from which all (100%) or substantially all (>90%, preferably >95% or >99%) of the bacteria were removed again, e.g. after fermentation. This definition, thus, encompasses cell-free medium (i.e. medium from which all or essentially all bacterial cells of the strain are removed, e.g. by centrifugation, filtration or other means), or fractions of such cell-free medium. Optionally, the cell-free medium may be treated in such a way that any potentially remaining bacterial cells are killed.

Various fermentation media are suitable according to the invention, such as (but not limited to) e.g. firstly an industrial medium, in which the strain(s) is/are grown, and that is used as is or after concentration (e.g. drying) or after addition to another food base or product such as a yoghurt and/or milk or secondly milk or milk based or milk comprising media such as milk with addition of vitamins and/or peptones and/or minerals in which the strains are grown, alone or in combination with other LAB, and that is used directly or after concentration (e.g. drying), or after combining with another food base or product, such as a fruit preparation.

Alternatively, bacterial cells, or bacterial cells with medium (e.g. the fermentation broth), or fractions of such cell comprising medium (i.e. medium with said bacterial strain/s) may be used. The cells or the cell comprising medium may further comprise live or viable bacterial cells and/or dead or non-viable bacterial cells of the strain(s). The medium may thus be treated by, but not limited to, heating or sonication to kill part or all of the bacteria of the strain(s).

Also lyophilized, or frozen, bacteria and/or cell-free media (which may be concentrated) are encompassed herein.

Compositions comprising one or more bacterial strains as such and/or bacterial cells with medium and/or one or more cell-free media (or parts thereof) include pharmaceutical compositions, complete food or feed compositions, food- or feed supplements, nutraceutical compositions, and the like. The strains (as such or with culture medium) and/or cell-free media may be added at any time during the production process of the composition, e.g. they may be added to a food base at the beginning of the production process or they may be added to the final food product. “Food” refers to liquid, solid or semi-solid dietetic compositions, especially total food compositions (food-replacement), which do not require additional nutrient intake or food supplement compositions. Food supplement compositions do not completely replace nutrient intake by other means. Food and food supplement compositions are in a preferred embodiment fermented dairy products or dairy-based products, which are preferably administered or ingested enterally, preferably orally one or more times daily.

Alternatively, food and/or food supplement compositions may be non-dairy or dairy non fermented products (e.g. strains or cell-free medium in non fermented milk or in another food medium).

In another embodiment, strains or cell-free medium can be encapsulated and dispersed in a food (e.g. in milk) or non food medium.

Fermented dairy products can be made directly using the bacteria according to the invention in the production process, e.g. by addition to the food base, using methods known per se. In such methods, the strain(s) of the invention may be used in addition to the micro-organism usually used, and/or may replace one or more or part of the micro-organism usually used. For example, in the preparation of fermented dairy products such as yoghurt or yoghurt-based drinks, a bacterium of the invention may be added to or used as part of a starter culture or may be suitably added during such a fermentation. Optionally the bacteria may be inactivated or killed later in the production process.

Fermented dairy products include milk-based products, such as (but not limited to) deserts, yoghurt, yoghurt drinks, quark, kefir, fermented milk-based drinks, buttermilk, cheeses, dressings, low fat spreads, fresh cheese, soy-based drinks, ice cream, etc. Non-fermented dairy products may include ice cream, nutritional bars and dressings, and the like. Non-dairy products may include powdered beverages and nutritional bars, and the like. The products may be made using known methods, such as adding an effective amount of the strain(s) and/or cell-free culture medium to a food base, such as skimmed milk or milk or a milk-based composition and fermentation as known. Other food bases to which the (compositions comprising the) bacterial cells and/or cell-free culture medium may be added are meat, meat replacers or plant bases.

The composition may be solid, semi-solid or liquid. It may be in the form of a food product or food supplement, e.g. in the form of tablets, gels, powders, capsules, drinks, bars, etc.

The composition comprises an effective amount of the bacterial cells and/or cell-free media, whereby the effective amount is the amount which is suitable for achieving the satiety and/or satiation effect (in combination with the administration regime). The effective amount will vary, but can be easily determined by the skilled person. Appropriate ranges include at least about 1×10⁶ cfu, preferably between about 1×10⁶-1×10¹² cfu (colony forming units) per day, more preferably between about 1×10⁷-1×10¹¹ cfu/day, more preferably about 1×10⁸-5×10¹⁰ cfu/day, most preferably between 1×10⁹-2×10¹⁰ cfu/day, and/or cell-free media (concentrated or non-concentrated) from cultures grown to such levels.

It is understood that nutritional composition preferably comprises carbohydrates and/or proteins and/or lipids suitable for human and/or animal consumption. The compositions may or may not contain other bioactive ingredients, such as other (probiotic) strains, and prebiotics, which support the probiotic strains. In a preferred embodiment the food or feed composition is low in calories, i.e. it may be suitable for slimming or weight loss or weight control. Preferably the composition comprises low amounts of fat, or no fat. It may also comprise low amounts of protein and/or carbohydrates, or no proteins and/or carbohydrates.

A food supplement may comprise one or more carriers, stabilizers, prebiotics and the like. When using live or viable cells of the strain(s), the cells may be present in an encapsulated form in order to be protected against the stomach. For example the composition may be in the form of a powder packed in a sachet which can be dissolved in water, fruit juice, milk or another beverage.

A suitable dosage of bacterial strains and/or cell-free culture medium may also be used to make a pharmaceutical composition for treatment, therapy or prophylaxis of obesity, overweight, and overweight related diseases. Pharmaceutical compositions will usually be used for enteral (for example oral) or nasal/inhalation administration. Pharmaceutical compositions will usually comprise a pharmaceutical carrier in addition to the strain(s) of the invention. The preferred form depends on the intended mode of administration and (therapeutic) application. The pharmaceutical carrier can be any compatible, nontoxic substance suitable to deliver the strains(s) to the desired body cavity, e.g. the intestine of a subject. E.g. sterile water, or inert solids may be used as the carrier usually complemented with pharmaceutically acceptable adjuvants, buffering agents, dispersing agents, and the like. Pharmaceutical compositions may further comprise additional biologically or pharmaceutically active ingredients.

Food, food supplements, nutritive or pharmaceutical compositions will either be in liquid, e.g. a stabilised suspension of the strain(s), or in solid forms, e.g. a powder, or in semi-solid form or a cell-free media. E.g. for oral administration, the strain(s) or cell-free media can be administered in solid dosage forms, such as capsules, tablets, and powders, or in liquid dosage forms, such as elixirs, syrups, and suspensions. The strain(s) or cell-free media can be encapsulated in gelatine capsules together with inactive ingredients and powdered carriers, such as e.g. glucose, lactose, sucrose, mannitol, starch, cellulose or cellulose derivatives, magnesium stearate, stearic acid, sodium saccharin, talcum, magnesium carbonate and the like. The compositions may comprise additional components, such as proteins, carbohydrates, vitamins, minerals, trace elements, amino acids, other biologically or pharmaceutically active compounds, carriers, stabilisers, flavourings, other probiotic strains, prebiotics, and the like.

Also provided is a container, comprising a composition according to the invention, as described above. Such a container may be a package holding 1-100, and each individual value between 1 and 100, such as 1, 5, 10, 20, 30, 40, 50, 100 or more dosages in the form of tablets, capsules, powder, ampoules, sachets and the like. Likewise, packages may hold 1-200, 1-500 or more dosages. When (compositions comprising) different strains and/or cell-free culture media are to be co-administered, it is understood that containers may comprise separate dosages of each strain- and/or cell-free medium-comprising composition. Preferably the container comprises written labelling on the outside stating the beneficial effect or health effect of the composition. For example, the container may state that the composition is “for slimming”, “for weight loss”, “for weight control” or the like. The container may be of carton, glass, plastic, metal and the like. The container may also comprise tools suitable for administration of the composition if the composition is in liquid or powder form. Further, the container may comprise written instruction for use.

### Uses According to the Invention

The use of one or more strains according to the invention, one or more cell-free media derived from such strains and/or compositions comprising these, for the preparation of a pharmaceutical and/or nutritional (food- or food-supplement) composition for the treatment, control and/or prevention of obesity, overweight and overweight related diseases is provided herein.

Also, the use of one or more strains according to the invention, one or more cell-free media derived from such strains and/or compositions comprising these, for the preparation of a pharmaceutical and/or nutritional (food- or food-supplement) composition for reducing food intake and/or for enhancing satiety and/or satiation is provided.

The following non-limiting Examples describe the uses and methods according to the invention. Unless stated otherwise, the practice of the invention will employ standard conventional methods of molecular biology, pharmacology, immunology, virology, microbiology or biochemistry. Such techniques are described in Sambrook and Russell (2001) Molecular Cloning: A Laboratory Manual, Third Edition, Cold Spring Harbor Laboratory Press, NY, in Volumes 1 and 2 of Ausubel et al. (1994) Current Protocols in Molecular Biology, Current Protocols, USA and Remington's Pharmaceutical Sciences, Mack Publishing Company, Philadelphia, Pa., 17th ed. (1985), Microbiology: A Laboratory Manual (6th Edition) by James Cappuccino, Laboratory Methods in Food Microbiology (3^{rd} edition) by W. Harrigan (Author) Academic Press, all incorporated herein by reference.

### EXAMPLES

### 1. Materials and Method

### 1.1 Preparation of the Mammalian Cell Line in 96 Well-Plates

Cell line STC-1 was grown for 3-4 days in the 96 well-plates at 37° C. in D-MEM (Dulbecco's Modified Eagle Medium) with penicillin/streptomycin and calf serum, in an atmosphere of air supplemented with 5% CO₂ until they reached about 85% density (approximately 500 000 cells per well). The culture medium was removed, and the cells were washed with 200 μl HBSS (Hanks' Buffered Salt Solution)/well.

### 1.2 Preparation of Test Samples for Use in In Vitro Mammalian Cell-Line Assays

Bacterial strains (see below) were freshly grown in MRS medium (Mann-Rogosa-Sharpe) and the CFU (Colony Forming Units) was determined. Cell-free medium for use in the STC-1 stimulation assay was prepared by separation of the bacterial strains from the growth medium by centrifugation. The cell-free medium was diluted 1:10 with HBSS and used in the STC-1 stimulation assay.

Skimmed milk, with addition of 0.5% casaminoacids (acid hydrolysed casein; Becton Dickinson and Company) (to stimulate bacterial growth), was fermented at 37° C. using different bacterial strains (see below) to cell numbers between 5×10⁷ and 5×10⁹ CFU/mL

Fermentation times varied per strain and were from about 15 to about 48 h until stationary phase was reached.

The fermented milks were used when they reached at least a pH of 5.5 and were then

1. adjusted to pH 4.35 by lactic acid; centrifuged to pellet the cells and coagulated proteins. Supernatant was collected, frozen and stored at −80° C. until use in the STC-1 cell culture stimulation. Before incubation with STC-1 cell cultures, the supernatants were diluted 10 times in HBSS buffer. The control in this type of experiments was milk acidified with lactic acid to pH 4.35 or HBSS buffer as such.
2. fermented milks with different end pH's were centrifuged to pellet the cells and coagulated protein, supernatants were collected, frozen and stored at −80° C. until use in the STC-1 cell culture stimulation. Before the incubation with STC-1 cells the supernatants were diluted 10 times in HBSS buffer.

Secreted CCK levels after incubation of STC-1 cell cultures with supernatants of fermented milks were compared with secreted CCK level stimulated by control milk acidified on comparable pH and/or by HBSS buffer as such.

### 1.3 Test for CCK Stimulation and Secretion by STC-1 Cell Line Cultures

In order to identify and/or isolate (probiotic) bacteria capable of stimulating (via the bacterial cells, or via the cell-free medium) gastrointestinal satiety hormone production, in vitro cell line assays were used.

200 μl of test solution (i.e. 200,000 bacterial cells, as it was the highest concentration which did not negatively affect the STC-1 cells, or bacterial cell culture supernatants in HBSS) was added to each well in 96-well plate and incubated for 2 h at 37° C. in presence of 5% CO₂. The “health” state of STC-1 cells after incubation with various test dilutions was checked by microscopic observation of each well.

After incubation, the test plates were centrifuged for 3 min., 1200 rpm to avoid presence of floating cells in the test samples. CCK levels were determined in the test samples by commercially available ELISA kits (Nuclilab, Ede, The Netherlands).

### LAB Strains Used

The following nine strains were used:

Lactobacillus acidophilus MUH 41 (CBS 121540)
Lactobacillus delbrueckii ssp bulgaricus MUH 192 (CBS 121543)
Lactobacillus casei ssp paracasei MUH 142 (CBS 121541)
Lactobacillus delbrueckii ssp bulgaricus MUH 190 (CBS 121542)
Lactobacillus salivarius 1502 (DSM 15794)
Lactobacillus acidophilus NCFM (PTA-4797)
Lactobacillus sakei 570 (DSM 15889)
Lactobacillus salivarius Ls-33 (PTA-4800)
Lactobacillus curvatus 853 (DSM 15793)

### 2. Results and Discussion

### 2.1 Fermented Milk Supernatants (Cell Free Medium)

Supernatants of L. casei ssp. paracasei 142, L. bulgaricus 190, L. bulgaricus 192, L. acidophilus 41, L. salivarius 1502, L. salivarius Ls-33 and L. acidophilus NCFM adjusted to the pH 4.35, stimulated higher CCK secretion compared to the control milk (acidified with lactic acid to pH 4.35) (Table 1).

TABLE 1
CCK secretion upon exposure of STC-1 cells to
fermented milk supernatants adjusted to pH 4.35

| | | CCK secretion - % |
|---|---|---|
| | CCK secretion - % | of control (milk acidified |
| | of basal secretion | with lactic acid to |
| Supernatant of strain | (HBSS) | pH 4.35) |
| L. casei ssp. paracasei | 530 | 217 |
| 142 | | |
| L. bulgaricus 190 | 663 | 271 |
| L. bulgaricus 192 | 525 | 215 |
| L. acidophilus 41 | 807 | 203 |
| Lactobacillus salivarius | 624 | 157 |
| 1502 | | |
| Lactobacillus acidophilus | 976 | 245 |
| NCFM | | |
| Lactobacillus salivarius | 240 | 190 |
| Ls-33 | | |

Samples prepared in unicate

Supernatants of the same strains (but without pH adjustment) also stimulated CCK secretion above the control values expected at that pH (Table 2).

TABLE 2
CCK secretion upon exposure of STC-1 cells
to fermented milk supernatants at the pH indicated
(not corrected with lactic acid)

| strain | Growth medium | pH | CCK (ng/mL) |
|---|---|---|---|
| Lactobacillus | Milk | 5.12 | 0.214 |
| bulgaricus 192 | | | |
| Lactobacillus | milk with addition | 4.36 | 0.171 |
| acidophilus 41 | of casaminoacids | | |
| Lactobacillus casei | Milk | 4.56 | 0.146 |
| ssp. paracasei 142 | | | |
| Lactobacillus | Milk | 4.73 | 0.133 |
| bulgaricus 190 | | | |
| Lactobacillus | milk with addition | 4.40 | 0.182 |
| salivarius 1502 | of casaminoacids | | |
| Lactobacillus | milk with addition | 4.72 | 0.144 |
| acidophilus NCFM | of casaminoacids | | |

pH dependency in milk acidified with lactate: CCK = 0.0836*pH-0.3002

TABLE 3
CCK induction by supernatants of LAB grown
in milk with addition of casoaminoacids.

| | CCK average | % of basal secretion |
|---|---|---|
| | (ng/ml) | (HBSS) |
| Control milk (acidified | 0.085 | 128% |
| with lactic acid to pH | | |
| 4.35) | | |
| L. bulgaricus 192 | 0.104 | 155% |
| L. acidophilus 41 | 0.194 | 290% |
| L. salivarius 1502 | 0.155 | 231% |
| L. acidophilus NCFM | 0.150 | 225% |

Values are means of three individual experiments.

Similar results were obtained using MRS as fermentation medium, instead of milk in similar assays.

2.2 Cell-Free Media of L. sakei 570 and L. curvatus 853 cultured in MRS

Data in Table 4 shows that the cell-free medium of L. sakei 570 and L. curvatus 853 strongly stimulates both the secretion of CCK compared to HBSS and MRS.

TABLE 4
CCK induction by cell-free medium of L. sakei 570
and L. curvatus 853 (results from duplicates)

| | | CCK secretion - % | |
|---|---|---|---|
| | Growth | of basal secretion | CCK secretion - % |
| strain | medium | (HBSS) | of MRS |
| Lactobacillus sakei | MRS | 410 | 195 |
| 570 | | | |
| Lactobacillus | MRS | 300 | 140 |
| curvatus 853 | | | |

### Conclusion

The supernatant (cell free fermentation medium) of all 9 strains stimulated CCK production and secretion in STC-1 cells.

### 3. Validation Experiment in Humans

The effect of a satiety inducing Lactobacillus strain, for example L. acidophilus 41 is assessed in a randomized, double-blind, placebo-controlled, within subject crossover trial in humans. Thirty volunteers (women, age 20-59, BMI 24-29 kg/m²) participate. Each subject is studied on two occasions, with a one week interval. The subjects are asked to fast from 20:00 h on the evening before the test day.

A test product is prepared as follows: Skimmed milk with 0.5% milk protein hydrolysate (DMV International) is heated to 95° for 5 min and cooled to 37°. Next it is inoculated with freeze-dried L. acidophilus MUH41 (10⁸ cfu/ml). Fermentation is performed at 37° C. until a pH of 5.4 is reached. 10% of a strawberry preparation with sugar and citric acid is added and additional sugar syrup is added to a final concentration of 7% sugar and a pH of 4.4. The preparation is cooled to 4° C. and stored in 350 ml sealed beakers. A placebo is made by using glucono-delta-lactone to acidify the milk with protein hydrolysate, the post treatment is the same as for the test product. The participants (15 placebo, 15 test product) consume 1 beaker of yoghurt at 9:00 h. Hunger and satiety scores are measured using a Visual Analogue Score (VAS). At 12:00 h a lasagna meal is presented and the participants are asked to eat until they are comfortably full. The participants on test product report less hunger feelings and eat less from the lasagna as compared to the participants on placebo.

Similar experiments are performed with other described strains e.g. L. acidophilus NCFM and L salivarius 1502.

### 4. Fermented Milk-Based Products

4.1. Product with L. acidophilus MUH41 as Single Strain

See section 3 above, human validation.

4.2. Product with Lactobacillus casei ssp paracasei MUH142.

Skimmed milk with 0.5% milk protein hydrolysate (DMV International) was (after heating to 95° and cooling to 37°) inoculated with freeze-dried Lactobacillus casei ssp paracasei MUH142 (10⁸ cfu/ml). Fermentation was performed at 37° C. until a pH of 5.3 was reached. Lactic acid was added to a pH of 4.4. This preparation was mixed in a 1:1 ratio with a commercially available fruit 0% fat yoghurt with acesulfame and aspartame as sweeteners (Campina Optimel Peach).

A similar product is made with other described individual strains.

4.3. Co-Fermentation of Lactobacillus delbrueckii ssp bulgaricus MUH192 and a Streptococcus thermophilus

A starter was produced from MUH 192 on a whey permeate medium known by the expert at 37°. At pH 4.5 cells were harvested and concentrated 20-50 times by centrifugation. Concentrated cell suspension was pelletized in liquid nitrogen and stored below −40 upon use. Yoghurt milk was made of milk and milk powder to obtain a protein content of 5% and was heated for 5 minutes at 95° C. before cooling to fermentation temperature of 37° C. This milk was inoculated with a commercial Streptococcus thermophilus strain with recommended dosage in combination with the prepared MUH192 (0.05%) followed by fermentation at 37° C. The fermentation was stopped at pH 4.5, structured and filled at 20° C. into appropriate packaging. Final cell counts of the strains can be validated with the plate-count technique described earlier.

A similar product is made with other described individual strains.

4.4. Yoghurt with L. acidophilus MUH41

A starter was produced from MUH41 on basic whey permeate medium at 37°. At pH 4.5 cells were harvested and concentrated 20-50 times by centrifugation. Concentrated cell suspension was palletized in liquid nitrogen and stored below −40 upon use. Yoghurt milk was made of milk and milk powder to obtain a protein content of 5% and was heated for 5 minutes at 95° C. before cooling to fermentation temperature of 37° C. This milk was inoculated with a commercially available yoghurt starter (Streptococcus thermophilus and Lactobacillus bulgaricus) with recommended dosage in combination with the prepared MUH41 (0.05%) followed by fermentation at 37° C. The fermentation was stopped at pH 4.5, structured and filled at 20° C. into appropriate packaging.

Similar or equivalent dairy products are being performed with other described strains, e.g. with L. acidophilus NCFM and/or L. salivarius 1502.

### 5. Stability in Product

The survival of the selected Lactobacillus strains in a yoghurt-like product environment under refrigeration conditions is assessed. Lactobacillus pre-cultures are grown overnight in MRS. Protease positive Streptococcus thermophilus cultures used to prepare yoghurt like products are pre-cultured overnight in milk treated for 10 minutes at 115° C. S. thermophilus pre-cultures are inoculated at a 0.1% density together with a 1% inoculum of the Lactobacillus pre-cultures in pasteurized (5 minutes at 85° C.) skim milk (prepared from milk powder) that has been cooled to 37° C. Inoculated cultures are aliquoted in portions of 100 ml. Skimmed milk cultures are grown at 37° C. for 15 hours and after growth are stirred with a sterile pipette, cooled to 4° C. and stored in a refrigerator (4-6° C.). Lactobacillus culture densities are determined by colony forming unit per ml enumeration on MRS-agar plates, directly after growth and cooling (timepoint 0), and after 2, 8, 15, and 28 days of storage in the refrigerator. In addition to viable cell density, the pH of the yoghurt like products is measured at the same time points.

The results generally reveal clear differences between the different Lactobacillus strains. However, MUH41, MUH142, MUH190, MUH192, L. acidophilus NCFM, L salivarius 1502, L salivarius Ls 33, L. curvatus 853 and L. sakei 570 display very good survival during the refrigerated storage. The viability of these strains over time appears stable and does not decrease or increase significantly (more than one log unit) during the period of refrigerated storage of 4 weeks.

1. A composition for inducing or enhancing satiety and/or satiation in a subject, comprising bacteria of at least one bacterial strain, or cell-free culture medium obtained from culturing said bacteria, wherein said strain is a strain of Lactic Acid Bacteria (LAB),
which bacteria or cell-free medium stimulates production of cholecystokinin (CCK) in an in vitro assay of cells of cell line STC-1.

2. The composition according to claim 1, comprising at least part of the culture medium.

3. The composition according to claim 1, wherein said culture medium comprises or consists of milk.

4. The composition according to claim 1, wherein said culture medium is free of live or viable bacteria of said strain.

5. The composition according to claim 1, wherein said composition is a fermented dairy product.

6. The composition according to claim 1, wherein said bacterial strain is of one of the following species: Lactobacillus delbrueckii, Lactobacillus salivarius, Lactobacillus curvatus or Lactobacillus sakei.

7. The composition according to claim 1, wherein said bacterial strain is of the species Lactobacillus acidophilus.

8. The composition according to claim 1 comprising bacteria of at least two bacterial strains, or cell-free culture media of said bacteria of said at least two strains, wherein bacteria of each of said strains or said cell-free culture media stimulates production of CCK in said.

9. The composition according to claim 1, wherein said bacterial strain is one or more of the following strains deposited in Centraalbureau voor Schimmelcultures, the ATCC, or the Deutsche Sammlung von Mikroorganismen and Zellkulturen, and has the indicated Accession number: CBS 121540 (L. acidophilus MUH 41), CBS 121543 (L. delbrueckii ssp bulgaricus MUH 192), CBS 121541 (L. casei ssp paracasei MUH 142), CBS 121542 (L. delbrueckii ssp bulgaricus MUH 190), PTA-4797 (L. acidophilus NCFM), PTA-4800 (L. salivarius 33), DSM 15794 (L. salivarius 1502), DSM 15793 (L. curvatus 853), DSM 15889 (L. sakei 570), or a derivative or replicate thereof.

10. The composition according to claim 1 in the form of a pharmaceutical, food or food supplement composition.

11. A method for identifying and/or isolating bacteria which are capable of inducing or enhancing satiety and/or satiation, comprising the steps of:
(a) culturing cells of one or more bacterial strains in a culture medium,
(b) collecting the bacterial cells, the bacterial cells with culture medium, and/or the culture medium free of cells of step (a),
(c) adding the bacterial cells, the bacterial cells with medium, and/or the cell-free medium collected in (b) to a culture comprising cells of a mammalian cell line,
(d) measuring the amount of CCK produced by the mammalian cells, and
(e) identifying which bacterial strain or cell-free medium induced an increase in CCK production in step (d) compared to control mammalian cells of said cell line.

12. The method according to claim 11, wherein said culture medium comprises milk.

13. A bacterial strain identified by the method of claim 11.

14. A bacterial strain deposited in the Centraalbureau voor Schimmelcultures under Accession number CBS 121540 (L. acidophilus MUH 41), CBS 121543 (L. delbrueckii ssp bulgaricus MUH 192), CBS 121541 (L. casei ssp paracasei MUH 142), CBS 121542 (L. delbrueckii ssp bulgaricus MUH 190), or a derivative or replicate thereof.

15.-16. (canceled)

17. A method of preparing a food, food supplement or feed composition that enhances or induces satiety and/or satiation, comprising the steps of:
a) identifying a bacterial strain that induces production of CCK in an in vitro assay using cell line STC-1,
b) producing said food, food supplement or feed product with bacteria of said strain identified in step (a), or with cell-free culture medium obtained from culturing said bacteria.

18. The method according to claim 17, wherein the producing step (b) comprises fermentation with said bacteria.

19. The method according to claim 17, wherein said food or food supplement composition is a yoghurt, a yoghurt drink, kefir, a fermented milk-based drink, a dessert, buttermilk, cheese, a dressing, ice cream, a low fat spread, a soy-based drink, a nutritional bar or a powdered beverage.

20. The method according to claim 17, wherein said strain is one of the following deposited strains and has the indicated Accession number: CBS 121540 (L. acidophilus MUH 41), CBS 121543 (L. delbrueckii ssp bulgaricus MUH 192), CBS 121541 (L. casei ssp paracasei MUH 142), CBS 121542 (L. delbrueckii ssp bulgaricus MUH 190), PTA-4797 (L. acidophilus NCFM), PTA-4800 (L. salivarius 33), DSM 15794 (L. salivarius 1502), DSM 15793 (L. curvatus 853) and DSM 15889 (L. sakei 570), or a derivative or replicate thereof.

21. A method for reducing food intake and/or enhancing satiety and/or satiation, or for controlling and/or preventing obesity, overweight or an overweight-related disease in a subject, comprising administering or feeding to the subject the composition according to claim 1,
thereby reducing said food intake and/or enhancing said satiety, or controlling and/or preventing said obesity, overweight, or said overweight-related disease

22. The method according to claim 21, wherein said bacterial strain is one or more of the following deposited strains and has the indicated Accession number: CBS 121540 (L. acidophilus MUH 41), CBS 121543 (L. delbrueckii ssp bulgaricus MUH 192), CBS 121541 (L. casei ssp paracasei MUH 142), CBS 121542 (L. delbrueckii ssp bulgaricus MUH 190), PTA-4797 (L. acidophilus NCFM), PTA-4800 (L. salivarius 33), DSM 15794 (L. salivarius 1502), DSM 15793 (L. curvatus 853), DSM 15889 (L. sakei 570), or a derivative or replicate thereof.

23. The composition of claim 5 wherein said dairy product is produced by fermentation with bacteria of said strain.

24. The composition of claim 6 wherein the bacterial strain is Lactobacillus salivarius, strain DSM15794

25. The composition of claim 7 wherein the bacterial strain is Lactobacillus acidophilus strain CBS121540,

26. A method for preparing a composition that, when administered to a subject, reduces food intake, induces or enhances satiety and/or satiation, treats or prevents obesity and/or promotes weight loss, comprising
(a) identifying and isolating said bacteria in accordance with the method of claim 11, and
(b) formulating said bacteria, said bacteria in culture medium, or said cell-free medium as said composition.
